(19) United States
(12) Patent Application Publication
(10) Pub. No.: US 2014/0039374 A1
(43) Pub. Date: Feb. 6, 2014

(54) Clog Detection in a Flow Control System

(71) Applicants: Cesario P. Dos Santos, Aliso Viejo, CA (US); Andrew D. Johnson, Tustin, CA (US)

(72) Inventors: Cesario P. Dos Santos, Aliso Viejo, CA (US); Andrew D. Johnson, Tustin, CA (US)

(21) Appl. No.: 13/565,953

(22) Filed: Aug. 3, 2012

(51) Int. Cl. A61F 9/00; A61M 1/00

(52) U.S. Cl. 604/9

(57) ABSTRACT

Disclosed herein are an apparatus, a system, and a method to monitor drainage from a first region of a patient to a second region. The apparatus comprises pressure sensors configured to measure the pressures of the first and second regions, a flow system to regulate drainage of fluid from the first region to the second region, a memory having a stored pressure drop across the flow system; and a processor associated with the memory and configured to compare the first pressure, the second pressure, and the stored pressure drop threshold and selectively generate control signals based on the comparisons. In some instances, the apparatus further comprises an alarm, and the processor is configured to generate the control signals that trip the alarm when a pressure drop exceeds the stored pressure drop threshold.

### BACKGROUND

The present disclosure relates generally to pressure/flow control systems and methods for use in treating a medical condition. In some instances, embodiments of the present disclosure are configured to be part of an IOP control system for the treatment of ophthalmic conditions.

Glaucoma, a group of eye diseases affecting the retina and optic nerve, is one of the leading causes of blindness worldwide. Most forms of glaucoma result when the intraocular pressure (IOP) increases to pressures above normal for prolonged periods of time. IOP can increase due to high resistance to the drainage of the aqueous humor relative to its production. Left untreated, an elevated IOP causes irreversible damage to the optic nerve and retinal fibers resulting in a progressive, permanent loss of vision.

The eye's ciliary body continuously produces aqueous humor, the clear fluid that fills the anterior segment of the eye (the space between the cornea and lens). The aqueous humor flows out of the anterior chamber (the space between the cornea and iris) through the trabecular meshwork and the uveoscleral pathways, both of which contribute to the aqueous drainage system. The delicate balance between the production and drainage of aqueous humor determines the eye's IOP.

FIG. 1 is a diagram of the front portion of an eye that helps to explain the processes of glaucoma. In FIG. 1, representations of the lens 110, cornea 120, iris 130, ciliary body 140, trabecular meshwork 150, Schlemm's canal 160, and the anterior chamber 170 are pictured. Anatomically, the anterior segment of the eye includes the structures that cause elevated IOP which may lead to glaucoma. Aqueous fluid is produced by the ciliary body 140 that lies beneath the iris 130 and adjacent to the lens 110 in the anterior chamber 170 of the anterior segment of the eye. This aqueous humor washes over the lens 110 and iris 130 and flows to the drainage system located in the angle of the anterior chamber 170. The angle of the anterior chamber 170, which extends circumferentially around the eye, contains structures that allow the aqueous humor to drain. The trabecular meshwork 150 is commonly implicated in glaucoma. The trabecular meshwork 150 extends circumferentially around the anterior chamber. The trabecular meshwork 150 seems to act as a filter, limiting the outflow of aqueous humor and providing a back pressure that directly relates to IOP. Schlemm's canal 160 is located beyond the trabecular meshwork 150. Schlemm's canal 160 is fluidically coupled to collector channels (not shown) allowing aqueous humor to flow out of the anterior chamber 170. The two arrows in the anterior segment of FIG. 1 show the flow of aqueous humor from the ciliary bodies 140, over the lens 110, over the iris 130, through the trabecular meshwork 150, and into Schlemm's canal 160 and its collector channels.

One method of treating glaucoma includes implanting a drainage device in a patient's eye. The drainage device allows fluid to flow from the interior chamber of the eye to a drainage site, relieving pressure in the eye and thus lowering IOP. These devices are generally passive devices and do not provide a smart, interactive control of the amount of flow through the drainage tube.

The system and methods disclosed herein overcome one or more of the deficiencies of the prior art.

### SUMMARY

In one exemplary aspect, the present disclosure is directed to an apparatus for treatment of a medical condition of a patient to monitor drainage from a first region of a patient to a second region. The apparatus comprises a first pressure sensor, a second pressure sensor, a flow system, a memory, and a processor. The first pressure sensor is configured to measure a first pressure of the first region. The second pressure sensor is configured to measure a second pressure of the second region. The flow system regulates drainage of fluid from the first region to the second region. The memory has a stored pressure drop threshold across the flow system, and the processor is associated with the memory. The processor is configured to compare the first pressure, the second pressure, and the stored pressure drop threshold and selectively generate control signals based on the comparisons.

In one aspect, the processor is configured to calculate a pressure differential between the first pressure and the second pressure and compare the pressure differential with the stored pressure drop threshold to determine whether the pressure differential is within an acceptable range.

In another aspect, the apparatus further comprises an alarm, wherein the control signals trip the alarm, the processor being configured to generate the control signals when the processor determines that the pressure differential is not within an acceptable range.

In another aspect, the apparatus further comprises an accessory device apart from the flow system, wherein the alarm is carried on the accessory device.

In another aspect, the stored pressure drop threshold comprises an upper pressure drop threshold and a lower pressure drop threshold.

In another exemplary aspect, the present disclosure is directed to a control system for treatment of an ocular condition of a patient to ensure drainage from an anterior chamber of the eye to a drainage location. The control system comprises a first pressure sensor, a second pressure sensor, a flow system, a memory, and a processor. The first pressure sensor is configured to detect a pressure representative of an anterior chamber of the eye. The second pressure sensor is configured to detect a pressure representative of the drainage location. The flow system regulates drainage of fluid from the anterior chamber to the drainage location. The memory has a stored pressure drop threshold across the flow system, the stored pressure drop threshold comprising an upper pressure drop threshold and a lower pressure drop threshold, and the processor is associated with the memory. The processor is configured to compare the first pressure, the second pressure, and the stored pressure drop threshold and selectively generate control signals based on the comparisons. In one aspect, the control system further comprises an alarm, wherein the control signals trip the alarm, and the processor is configured to generate the control signals when a pressure drop across the flow system is not within an acceptable range defined by the upper pressure drop threshold and the lower pressure drop threshold.

In another aspect, the control system further comprises an accessory device apart from the flow system, wherein the alarm is carried on the accessory device.

In another exemplary aspect, the present disclosure is directed to a method comprising: storing a pressure drop threshold in a memory, the pressure drop threshold comprising an upper pressure drop threshold and a lower pressure drop threshold, measuring a first pressure representative of the a region of an eye, measuring a second pressure representative of a second region of an eye, communicating the first and second pressures to a processor, calculating a pressure differential between the first and second pressures with the processor, comparing the pressure differential and the stored pressure drop threshold, and activating an alarm when the pressure differential exceeds the upper pressure drop threshold or when the pressure differential is less than the lower pressure drop threshold.

In one aspect, the method comprises storing a predetermined time interval in the memory, and evaluating if the pressure differential exceeds the upper pressure drop threshold or is less than the lower pressure drop threshold over the predetermined time interval.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram of the front portion of an eye.

FIG. 2 is a block diagram of an exemplary IOP control system according to the principles of the present disclosure.

FIG. 3 is a schematic diagram of an exemplary implant including the IOP control system of FIG. 2 disposed on an eye according to the principles of the present disclosure.

FIG. 4 is a schematic diagram of the exemplary implant shown in FIG. 3 including various clogs in the IOP control system.

FIG. 5 is a flow chart of an exemplary method of indicating a problem with the IOP control system, such as a clog, according to one embodiment consistent with the principles of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately. For simplicity, in some instances the same reference numbers are used throughout the drawings to refer to the same or like parts.

The present disclosure is directed to clog detection within a flow control system for treating a medical condition, such as glaucoma. In one aspect, the system adjusts IOP by regulating fluid drainage through an implant such as a glaucoma drainage device (GDD). The system directs fluid drainage from the anterior chamber of an eye to a drainage site through a drainage tube. In one aspect, the system includes stored pressure drop thresholds across the flow control system that are generally predetermined based on the structure of the flow control system. The system monitors the actual pressure difference between the anterior chamber and the drainage site to ensure that it lies within the stored pressure drop thresholds when the flow control system is in an open condition. If not, the system alerts the user and/or the health care provider to a possible problem in the system preventing the drainage of fluid from the anterior chamber to the drainage site. Possible problems include, by way of non-limiting example, a clog within the system or a ruptured drainage site. If a problem is detected, the flow control system may power down to conserve energy. Prompt awareness on the part of the user and/or the health care provider to an ineffective and/or clogged flow control system may result in better treatment, less complications, a more consistent recovery, and ultimately a better patient outcome.

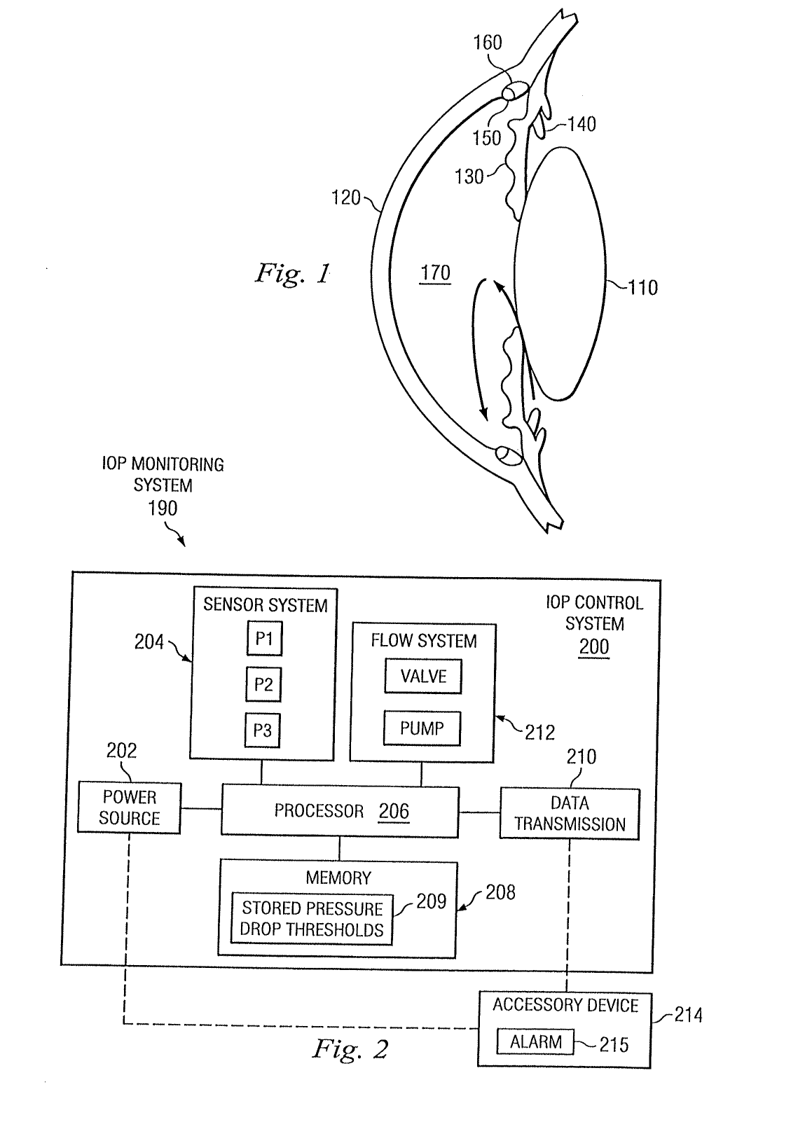

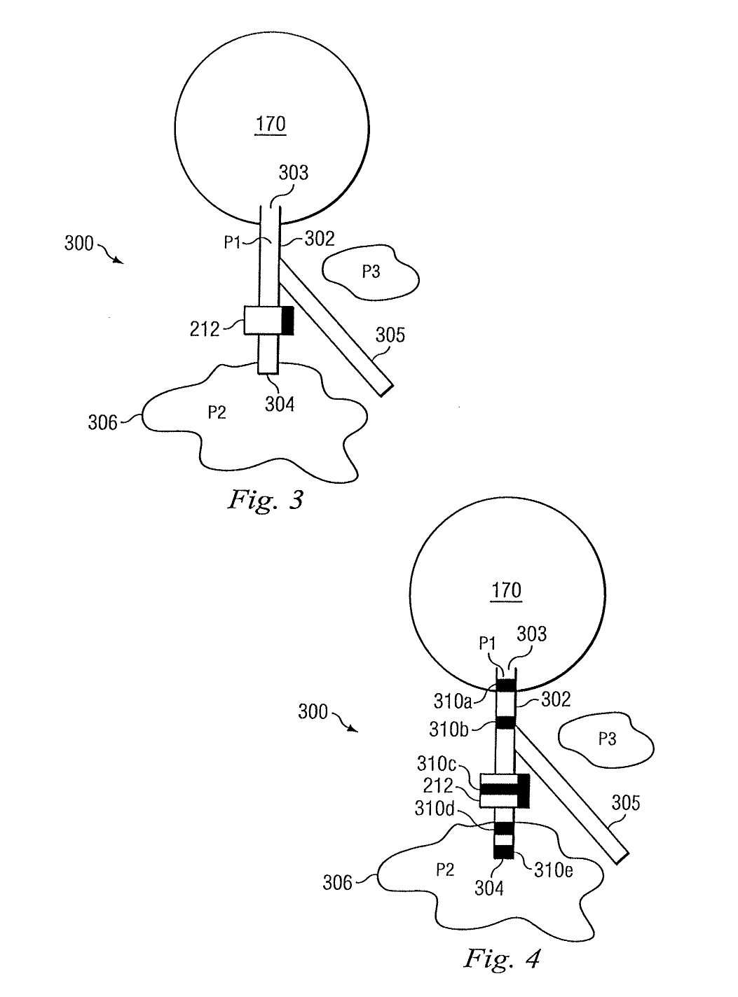

FIG. 2 is a block diagram of an exemplary IOP monitoring system 190. The IOP monitoring system 190 includes an IOP control system 200 that is usable as a part of an implant 300 (shown in FIG. 3) in an eye of a patient for the treatment of glaucoma or other conditions and includes an accessory device 214. In FIG. 2, the IOP control system 200 includes a power source 202, an IOP sensor system 204, a processor 206, a memory 208, a data transmission module 210, and a flow system 212.

The power source 202 is typically a rechargeable battery, such as a lithium ion or lithium polymer battery, although other types of power sources may be employed, such as a capacitive bank. In addition, any other type of power cell is appropriate for power source 202. Power source 202 provides power to the system 200, and more particularly to processor 206. In one embodiment, the power source can be recharged via inductive coupling such as an RFID link or other type of electromagnetic coupling.

The IOP sensor system 204 includes pressure sensors P1, P2, and P3, which are distributed to detect pressures at different locations about the IOP control system 200. The IOP sensor system 204 will be described in further detail below.

The processor 206 is typically an integrated circuit with power, input, and output pins capable of performing logic functions. In various embodiments, the processor 206 is a targeted device controller. In such a case, the processor 206 performs specific control functions targeted to a specific device or component, such as the data transmission module 210, the power source 202, the sensor system 204, the flow system 212, or the memory 208. In other embodiments, the processor 206 is a microprocessor. In such a case, the processor 206 is programmable so that it can function to control more than one component of the device and/or perform calculations to evaluate the status or functionality of various components of the device. For example, in one embodiment, the processor 206 is able to read data from the IOP sensor system 204 and perform calculations using the data to ensure that the flow system 212 is performing properly. In other cases, the processor 206 is not a programmable microprocessor, but instead is a special purpose controller configured to control different components that perform different functions.

The memory 208 is typically a semiconductor memory such as RAM, FRAM, or flash memory. The memory 208 interfaces with the processor 206. As such, the processor 206 can write to and read from the memory 208. For example, the processor 206 can be configured to read data from the IOP sensor system 204 and write that data to the memory 208. In the embodiments shown, stored pressure drop thresholds 209 across the flow system 212 are stored in the memory 208 for access, calculation, and comparison by the processor 206. The processor 206 is also capable of performing other basic memory functions, such as erasing or overwriting the memory 208, detecting when memory 208 is full, and other common functions associated with managing memory.

The data transmission module 210 may employ any of a number of different types of data transmission. For example, the data transmission module 210 may be an active device such as a radio. The data transmission module 210 may also be a passive device such as the antenna on an RFID tag. In one embodiment, an RFID tag includes the memory 208 and the data transmission module 210 in the form of an antenna. An RFID reader can then be placed near the system 200 to write data to or read data from the memory 208. Therefore, data such as the stored pressure drop thresholds 209 across the flow system 212 may be transmitted to the memory via the data transmission module, along with any selection of or adjustment to the stored pressure difference. Other types of data that can be stored in the memory 208 and transmitted by the data transmission module 210 include, but are not limited to, IOP measurement data, power source data (e.g. low battery, battery defect), speaker data (warning tones, voices), pressure sensor data (IOP readings, calculations), time stamp data, IOP regulation setpoint (or predetermined IOP setpoint or IOP target value), and the like. In some embodiments, the data transmission module 210 may be activated to communicate data to the accessory device 214.

The accessory device 214 is an external device such as, by way of non-limiting example, a PDA, cell phone, computer, wrist watch, custom device exclusively for this purpose, remote accessible data storage site (e.g. an internet server, email server, text message server), or other electronic device. For example, after a patient has undergone glaucoma surgery and had the flow control system 200 implanted, the accessory device 214 may be carried by the patient or periodically positioned near the patient. The processor 206 can read and compare pressure measurements made by the implanted sensor system 204. If the processor 206 reads data indicative of an undesirable and/or unsafe condition while the flow control system is open, such as, by way of non-limiting example, a clog or blockage in the IOP control system 200, the data transmission module 225 can transmit the relevant data to the accessory device 214, which utilizes an electronic notification or alarm 215 to alert the patient to the undesirable and/or unsafe condition. In other embodiments, the data transmission module 225 may regularly transmit data from the IOP sensor system 204 to the accessory device 214, which is able to perform calculations using the data to ensure that the IOP control system 200 is performing properly. In such embodiments, if the accessory device 214 determines that the presence of an undesirable and/or unsafe condition, the alarm 215 is activated to alert the patient to the condition.

The alarm 215 may comprise a visual, tactile, and/or audible signal that alerts the patient to a potential problem with the IOP control system 200 and/or the flow system 212. By way of non-limiting example, in some instances, the problem may comprise a clog or blockage in their implant. In other instances, the problem may comprise a ruptured drainage site, such as a ruptured bleb. In other instances, the alarm 215 may signal a correction of the patient's underlying IOP elevation, and an end to the need for operation of the IOP control system 200. The alarm 215 may spur the patient to seek immediate medical attention to correct the problem, thereby limiting the potential complications of prolonged exposure to a malfunctioning IOP control system. For example, an alarm alerting a patient to a clogged GDD may prompt the patient to seek immediate medical attention, thereby limiting the exposure of the optic nerve to elevated IOP pressures and increasing the probability of limiting further progression of their glaucoma.

In one embodiment, the accessory device 214 comprises a personal electronic device that uploads relevant data to a remote accessible data storage site (e.g., an internet server, email server, text message server) via wired or wireless communication. Information may be uploaded to a remote accessible data storage site so that it can be viewed in real time, for example, by healthcare professionals that are not present at the patient's side. Thus, both the patient and his or her healthcare professionals may be alerted to a problem with the IOP control system, such as a potential blockage or clog within the IOP control system or a ruptured drainage site, prior to the patient's next scheduled examination.

In one embodiment, the accessory device 214 comprises a personal electronic device used for both recharging of the power source 202 and data collection from the sensor system 204. Such an embodiment allows the step of checking the functionality of the implantable control system 200 to be integrated with the patient's routine steps of recharging the implant 300 and collecting data from the implant 300. Therefore, when the patient routinely recharges their implant 300 or collects data from the implant 300, the patient can also check the functional status of the implantable control system 200 by observing whether the alarm 215 has been activated or not. For example, in instances where the patient routinely recharges and collects data from the implant 300 every day, the accessory device 214 will also provide the patient with a specific daily reminder of the functional state of their implant, i.e., depending upon whether the alarm 215 is activated (indicating a potential problem) or not. If the alarm 215 has been activated, the patient will be alerted to a potential problem within the IOP control system 200, thereby prompting the patient to seek immediate medical attention, which may reduce the complications arising from prolonged exposure to an undesirably elevated IOP.

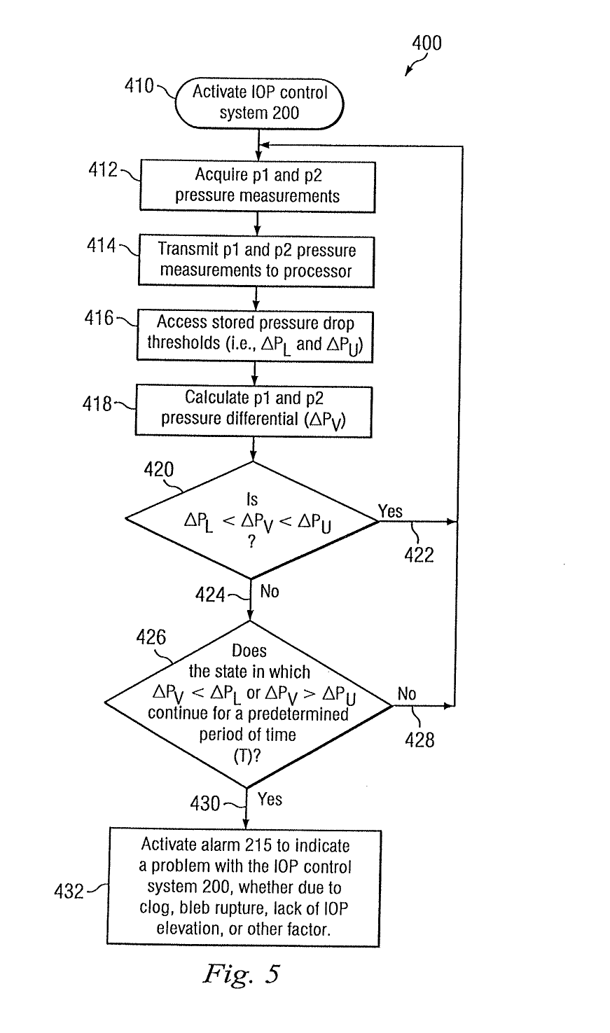

The pressure/flow system 212 may include components or elements that control pressure by regulating the amount of drainage flow. In the example shown, the flow system 212 includes a valve and a pump. The flow system may include any number of valves and any number of pumps, or may not include a pump or may not include a valve. In some embodiments, the flow system 212 is an active system that is responsive to signals from the processor 206 to increase flow, decrease flow, or to maintain a steady flow as a function of pressure differentials across the valve system. In one embodiment, it does this by maintaining a valve setting at a consistent setting, or increasing or decreasing the amount that the valve is open. In such embodiments, as described further below is relation to FIG. 5, an activated alarm 215 may indicate a resolution of the patient's elevated IOP, which may necessitate removal or deactivation of the IOP control system. The IOP sensor system 204 is described below with reference to FIG. 3.

FIG. 3 is a diagram of the exemplary IOP control system 200 as a part of the implant 300 implanted within an eye of a patient. In this example, the implant 300 includes a drainage tube 302 and a divider 305 associated with components of the control system 200. For example, the flow system 212 and the pressure sensors of the IOP sensor system 204 are identified in FIG. 3. In particular, the exemplary IOP sensor system 204 includes three pressure sensors, P1, P2, and P3 (also shown in FIG. 2). Pressure sensor P1 is located in or is in fluidic communication with the anterior chamber (labeled 170), pressure sensor P2 is located at a drainage site (e.g., 306 in FIG. 3) that may be in the subconjunctival space, and pressure sensor P3 is located remotely from P1 and P2 in manner to measure atmospheric pressure. In some embodiments, pressure sensor P1 is located in a lumen or tube that is in fluid communication with the anterior chamber.

Pressure sensors P1, P2, and P3 can be any type of pressure sensors suitable for implantation in the eye. They each may be the same type of pressure sensor, or they may be different types of pressure sensors. For example, pressure sensors P1 and P2 may be the same type of pressure sensor (implanted in the eye), and pressure sensor P3 may be a different type of pressure sensor (in the vicinity of the eye).

The IOP control system 200 responds to the pressure differentials between the pressures sensed by sensors P1, P2, and P3 to control the flow system 212 and thereby throttle the flow rate of aqueous humor through the drainage tube 302 to control IOP. In some embodiments, the various pressure differentials across the pressure areas sensed by P1, P2, and P3 (P1-P2, P1-P3, P2-P3) drive the flow system 212 and dictate the valve position or pump state to throttle the flow rate of aqueous humor through the drainage tube 302 without requiring external power at the flow system 212 to control IOP.

The drainage tube 302 drains aqueous humor from the anterior chamber 170 of the eye to the drainage location 306, which may be placed at any of numerous locations within the eye. For example, some tubes are arranged to shunt aqueous from the anterior chamber 170 to the subconjunctival space thus forming a bleb under the conjunctiva or alternatively, to the subscleral space thus forming a bleb under the sclera. Other tube designs shunt aqueous humor from the anterior chamber to the suprachoroidal space, the supraciliary space, the juxta-uveal space, or to the choroid, forming blebs in those respective locations. In other applications, the drainage tube shunts aqueous humor from the anterior chamber to Schlemm's canal, a collector channel in Schlemm's canal, or any of a number of different blood vessels like an episcleral vein. In some examples, the drainage tube even shunts aqueous humor from the anterior chamber to outside the conjunctiva. Each of these different anatomical locations to which aqueous is shunted is an example of a drainage location 306. Other examples of a drainage location 306 include, but are not limited to: a subconjunctival space, a suprachoroidal space, a subscleral space, a supraciliary space, Schlemm's canal, a collector channel, an episcleral vein, and an uveo-scleral pathway.

The flow system 212 throttles the flow of aqueous humor through the tube 302, from a drainage tube inlet 303 to a drainage tube outlet 304. In some instances, the flow system 212 throttles the flow of aqueous humor through the tube 302 as a function of a pressure differential. In the embodiment shown, the pressure sensor P1 measures the pressure in the tube 302 upstream from the flow system 212 and downstream from the anterior chamber 170. In this manner, pressure sensor P1 measures the pressure in the anterior chamber 170. The expected measurement discrepancy between the true anterior chamber pressure and that measured by P1 when located in a tube downstream of the anterior chamber (even when located between the sclera and the conjunctiva) is very minimal. For example, Poiseuille's law for pipe flow predicts a pressure drop of 0.01 mmHg across a 5-millimeter long tube with a 0.300 millimeter inner diameter for a flow rate of 3 microliters per minute of water. Therefore, because there is almost no pressure difference between the anterior chamber 170 and the interior of the tube 302 that is in fluid contact with the anterior chamber 170, the pressure sensor P1 effectively measures the pressure of the anterior chamber 170.

Pressure sensor P2 is located at the drainage site 306. As such, pressure sensor P2 may be located in a pocket, such as a bleb, that generally contains aqueous or in communication with such a pocket, via a tube for example, and is in a wet location 306. The drainage site 306 may be, for example, in a subconjunctival space, a suprachoroidal space, a subscleral space, a supraciliary space, Schlemm's canal, a collector channel, an episcleral vein, and an uveo-scleral pathway, among other locations in the eye.

In some embodiments, the divider 305 acts as a barrier that separates the pressure sensor P3 from the pressure sensor P2. In some embodiments, the system includes other barriers that separate the sensors P1, P2, and P3. These barriers may be elements of the system itself. In FIG. 3, the pressure sensor P3 is physically separated from the pressure sensor P2 by the divider 305. The divider 305 is a physical structure that separates the drainage area 306 from the isolated location of P3. In some embodiments, the divider 305 separating anterior chamber pressure sensor P1 and the drainage site pressure sensor P2 includes physical components of the flow system 212, such as parts of a housing. Note that the divider 305 may take many forms, such as, but not limited to, a tube connected to the sensor P3 that is routed to a site representative of atmospheric pressure (and away from and fluidly independent of the drainage site). The drainage site sensor P2 may then reside on the IOP control system 200 in direct contact with the drainage site.

Generally, IOP is a gauge pressure reading—the difference between the absolute pressure in the eye (as measured by sensor P1) and atmospheric pressure (as measured by sensor P3). Atmospheric pressure, typically about 760 mm Hg, often varies in magnitude by 10 mmHg or more depending on weather conditions or indoor climate control systems. In addition, the effective atmospheric pressure can vary significantly—in excess of 300 mmHg—if a patient goes swimming, hiking, riding in an airplane, etc. Such a variation in atmospheric pressure is significant since IOP is typically in the range of about 15 mm Hg. Thus, for accurate monitoring of IOP, it is desirable to have pressure readings for the anterior chamber (as measured by sensor P1) and atmospheric pressure in the vicinity of the eye (as measured by sensor P3).

In one embodiment of the present invention, pressure readings are taken by pressure sensors P1 and P3 simultaneously or nearly simultaneously over time so that the actual IOP can be calculated (as P1-P3 or P1-f(P3), where f(P3) indicates a function of P3). The pressure readings of P1 and P3 (as well as P2) can be stored in the memory 208 by the processor 206. They can later be read from memory so that actual IOP over time can be interpreted by a healthcare professional.

In another embodiment of the present invention, pressure readings taken by the pressure sensors P1, P2, and P3 can be used to control a device that drains aqueous from the anterior chamber 170. For example, in some instances, the IOP control system 200 reacts to the pressure differential across P1 and P3 continuously or nearly continuously so that the actual IOP (as P1-P3 or P1-f(P3)) can be responded to accordingly.

In one exemplary aspect, the present disclosure is directed to a system that utilizes the pressure measurements from the sensors P1 and P2 and the stored pressure drop thresholds 209 to evaluate the functionality of the IOP control system 200 and alert the patient to potential problems with the flow control system 200. For example, a potential problem that can arise is the clogging of the implant 300, as indicated in FIG. 4. Clogs or blockages within the IOP control system 200 may arise from a variety of medical conditions, especially in the early postoperative period. For example, clogs within the drainage tube 302 or the flow system 212 may arise from blood, vitreous, fibrin, iris incarceration, or buildup of other cellular debris.

As described above, the IOP control system 200 includes the predetermined pressure drop thresholds 209 across the flow system 212. The pressure drop represents the difference in pressure between the pressures measured by P1 and P2, caused by the resistance to flow through the drainage tube 302 and the flow system 212. The stored pressure drop thresholds 209 are generally predetermined by the structural characteristics of the flow system 212 and the flow demands on the system. In particular, the pressure drop across the flow control system 212 is determined as a function of the flow rate through the drainage tube 302 and the geometry of the flow control system 212. For example, as the flow rate increases, the pressure drop across the flow control system 212 increases proportionately, and as the flow rate decreases, the pressure drop across the flow control system 212 decreases proportionately.

Thus, in a passive flow control system where the base IOP setpoint does not change, the stored pressure drop thresholds 209 define the bounds of a predetermined range of acceptable values for the pressure drop across the flow control system. In active flow control systems, however, where the base IOP setpoint may be intentionally manipulated, the pressure drop thresholds 209 across the flow system 212 may be adjusted to a degree. In some embodiments, the pressure drop threshold 209 may comprise a discrete pressure value instead of a list of values. In some instances, the stored pressure drop thresholds 209 may be selected by a health care provider for utilization by the processor 208. For example, in some embodiments, the healthcare provider may adjust the stored pressure drop thresholds 209 while activating the flow control system. In some embodiments, the stored pressure drop thresholds 209 may be modified by the healthcare provider throughout the use of the implanted system.

FIG. 4 illustrates a situation where the implant 300 contains one or more blockages or clogs 310 along the drainage path of the aqueous humor. As shown, the clogs 310 may be located anywhere along the drainage path of the aqueous humor extending from the inlet 303 to the outlet 304. For example, by way of non-limiting example, the clog 310a is located near the drainage tube inlet 303, the clog 310b is located within the drainage tube 302 and proximal to the flow system 212, the clog 310c is located within the flow system 212, the clog 310d is located within the drainage tube 302 and distal to the flow system 212, and clog 310e is located at the drainage tube outlet 304. Clogs within the drainage tube 302 and the flow system 212 increase the resistance to flow from the inlet 303 to the outlet 304, thereby increasing the pressure proximal to the clog (as measured by the sensor P1) and decreasing the pressure distal to the clog (as measured by the sensor P2).

In an exemplary embodiment, the IOP control system 200 disclosed herein can detect such clogs within the system by comparing the measured pressures from sensors P1 and P2 with each other and with the stored pressure drop threshold 209. A flow chart 400 shown in FIG. 5 represents an exemplary clog detection algorithm employed by the IOP control system 200. For ease of description, the pressure measured by sensor P1 will be referred to as pressure p1 and the pressure measured by sensor P2 will be referred to as pressure p2.

The relationship between pressures p1 and p2 while the valve system 212 is in an open condition is generally described by the following equation: the pressure drop across the valve system 212, ΔP_{V}, equals the difference between pressures p1 and p2 (i.e., ΔP_{V}=p1−p2). The stored pressure drops 209 comprise a lower pressure drop threshold 209a and an upper pressure threshold 209b. In other words, the acceptable range of pressure drop values is bounded by the lower pressure drop threshold 209a, ΔP_{L}, and the upper pressure drop threshold 209b, ΔP_{U}. Therefore, by utilizing the pressure sensors P1 and P2, and comparing the pressure drop ΔP_{V} with the stored pressure drop thresholds 209a and 209b (i.e., ΔP_{L} and ΔP_{U}), the IOP control system 200 may determine if a clog or blockage exists within the implant 300. For example, if the valve system 212 of the implant 300 is in an open condition and ΔP_{V}>ΔP_{U} or ΔP_{V}<ΔP_{L}, there may be a clog within the implant 300, either in the drainage tube 302 or in the flow system 212, that is preventing the flow of aqueous humor from the anterior chamber 170 to the drainage site 306, or the bleb has been breached and aqueous humor is draining freely from the bleb. In another situation where the pressure drop ΔP_{V} may lie outside of the range defined by ΔP_{L} and ΔP_{U}, p2 may be approaching p1 because the bleb is severely scarred or fibrosed. In some embodiments, the flow control system may track the length of time required for p1 to meet a target IOP value, and if the length of time exceeds a certain predetermined time, the system may be alerted to either failed valve actuation or a forming or formed blockage within the flow control system.

FIG. 5 shows a flow chart representing an exemplary method of detecting an abnormal pressure drop and alerting a patient and/or healthcare provider of the condition. The method may be carried out by the IOP monitoring system 190 shown in FIG. 2. The method begins at a step 410, where the IOP control system 200 is activated and begins to regulate flow of aqueous humor through the drainage tube 302 from the anterior chamber 170 toward the drainage site 306. At step 412, the pressure sensors P1 and P2 acquire pressure measurements p1 and p2 from their respective positions within the IOP control system 200. In particular, as shown in FIG. 3, the sensor P1 measures the pressure p1 representing the pressure of the anterior chamber 170 and the sensor P2 measures the pressure p2 of the drainage site 306. At step 414, the detected pressures p1 and p2 are transmitted or otherwise communicated from the sensors P1 and P2 to the processor 206.

At step 416, the processor 206 accesses the stored pressure drop thresholds 209 from the memory 208 for comparison with the measured pressures p1 and p2. As described above in relation to FIG. 2, the stored pressure drop thresholds 209 (i.e., pressure drop thresholds 209a, or ΔP_{L}, and 209b, or ΔP_{U}) are stored in the memory 208 for use in the exemplary clog detection algorithm or pressure comparison method depicted in the flow chart 400. The IOP control system 200 may include a selected set of stored pressure drop thresholds 209 preloaded into the memory 208. This pre-selected or predetermined set can be established during manufacturing of the IOP control system 200 or programmed into the memory 208 by the user (e.g., a healthcare professional), and represents a set of pressure drop thresholds corresponding to typical known operating conditions for the system 200.

In the pictured embodiment, the processor 206 performs the steps 418-438. In some embodiments, the pressure measurements from sensors P1 and P2 and the stored pressure drop threshold 209 are transmitted to the accessory device 214, which performs the steps 418-438.

The processor 206 is configured to calculate a pressure differential between the pressures p1 and p2 and to compare the pressure differential with the stored pressure drop thresholds 209a and 209b to determine whether the pressure differential is within an acceptable range. The acceptable range of pressure differentials may vary between different IOP control systems. In one example, the acceptable range of pressure differentials (ΔP_{V}) may range from 2 mmHg to 5 mmHg. Such a range is merely exemplary in nature, and it not intended to be limiting. In particular, the processor 206 may be configured to determine that the pressure differential is not within an acceptable range if the pressure differential ΔP_{V} between the pressures p1 and p2 is greater than the stored pressure drop threshold 209b, ΔP_{U}. For example, in some embodiments, the ΔP_{U}=20 mmHg. In one example where the valve has been actuated open for a significant amount of time, the stored pressure drop threshold ΔP_{U}=20 mmHg, the IOP setpoint=10 mmHg, p1=780 mmHg, p2=752 mmHg and p3=750 mmHg. In this example, the processor 206 would determine that the IOP (p1-p3)=30 mmHg, and p1-p2 (ΔP_{V})=28 mmHg. The high ΔP_{V} value (ΔP_{V}>ΔP_{U}) indicates that a possible blockage has occurred.

The processor 206 may be configured to determine that the pressure differential ΔP_{V} is not within an acceptable range if the pressure differential ΔP_{V} between the pressures p1 and p2 is less than the stored pressure drop threshold 209, ΔP_{L}. In another example, the stored threshold pressure drop ΔP_{L}=1 mmHg, the IOP setpoint=10 mmHg, p1=755 mmHg, p2=754.5 mmHg, and p3=750 mmHg. In this example, the processor 206 would determine that the IOP (p1-p3)=5 mmHg, and p1-p2 (ΔP_{V})=0.5 mmHg. The low ΔP_{V} value (ΔP_{V}<ΔP_{L}) indicates that a problem has occurred, and the very low IOP value of 5 mmHg (IOP<<IOP setpoint) indicates that the eye is in a state of hypotony. Such pressure differentials and pressures are merely exemplary in nature, and are not intended to be limiting.

In the pictured embodiment, at step 418, the processor 206 calculates the pressure differential ΔP_{V} between pressures p1 and p2, i.e., p1 minus p2, when the flow system 212 is in an open condition. At step 420, the actual pressure differential ΔP_{V} between pressures p1 and p2 is compared to the stored pressure drop thresholds 209a and 209b (i.e., ΔP_{L} and ΔP_{U}, respectively). The comparisons may be made by the IOP control system 200 (e.g., by the processor 206 or the accessory device 214) according to the stored pressure drop thresholds 209 installed into the memory 208 at manufacture or entered by the user. Theoretically, the user would be able to input any stored pressure drop thresholds 209a and 209b that fall within the capabilities of the system.

If the pressure differential ΔP_{V} between pressures p1 and p2 is within the acceptable range of pressure differentials defined by the stored pressure drop thresholds 209, then the implant 300 is likely draining aqueous fluid from the anterior chamber 170 (shown in FIG. 3) to the drainage site 306 as intended. Thus, if, at step 422, the pressure differential ΔP_{V} is within the acceptable range defined by the stored pressure drop thresholds 209, then the process returns to step 412. If, however, the pressure differential ΔP_{V} between pressures p1 and p2 is outside of the acceptable range of pressure differentials defined by the stored pressure drop thresholds 209 while the valve system 212 is in an open condition, the implant 300 may not be operating correctly, and in particular the drainage tube 302 and/or the valve system 212 may be clogged (as shown in FIG. 4). Thus, if, at step 424, the pressure differential ΔP_{V} between pressures p1 and p2 is outside the range of acceptable pressure differentials defined by the stored pressure drop thresholds 209, then the processor 206 progresses to step 426.

At step 426, the processor 206 queries whether this state (i.e., where ΔP_{V}<ΔP_{L} or ΔP_{V}>ΔP_{U}) continues for a predetermined amount of time, T. The predetermined time T may be installed into the memory 208 at manufacture or entered by the user. The determination at step 432 may be made by comparing a second set of measured pressures p1 and p2 after passage of the time T.

At step 426, the processor 206 essentially queries whether the disruption in flow through the implant 300 is a transient condition such as a transient, swiftly-resolved occlusion or an on-going condition such as a stationary clog within the drainage tube 302. If, at step 428, the processor 206 determines that this state of disrupted flow (i.e., where the pressure differential ΔP_{V} is outside of an acceptable range of pressure differentials as defined by the stored pressure differential thresholds 209) does not continue for the predetermined amount of time T while the flow system 212 is in an open condition, then the process returns to step 412. If, however, at step 430, the processor 206 determines that this state continues for the predetermined amount of time T while the flow system 212 is in an open condition, the processor 206 at step 432 generates control signals to activate the alarm 215, which alerts the user and/or healthcare professional to a potential problem with the IOP control system 200.

In some embodiments, the processor generates the control signals to activate the alarm if the pressure p1 does not change while the flow system is in an open condition for a time equal to or greater than the predetermined amount of time T. In some embodiments, the processor generates the control signals to activate the alarm if neither the pressure p1 nor the pressure differential ΔP_{V} between pressures p1 and p2 changes while the flow system is in an open condition for a time equal to or greater than the predetermined amount of time T.

In some embodiments, the flow control system 212 powers down when the alarm 215 is activated to conserve energy. In some embodiments, the processor 206 may be configured to stop supplying power to the flow control system 212 when the alarm 215 is activated, thereby powering OFF the system.

As described above, the problem may comprise a clog or blockage in the implant 300. In other instances, the problem may comprise a ruptured drainage site 306, such as a ruptured bleb. In other instances, the alarm 215 may signal a correction of the patient's underlying IOP elevation, and an end to the need for operation of the IOP control system 200. In some embodiments, the alarm 215 comprises a range of signal intensities that vary depending upon the severity of the problem (e.g., the severity of the pressure drop). By way of non-limiting example, in some embodiments, the alarm may comprise a series of visual signals such as lights where the number of lit lights reflects the severity of the problem. In other embodiments, the alarm may comprise an audible tone which increases in frequency or pitch with increasing severity of the problem. In other embodiments, the alarm may comprise a digital readout on the accessory device 214.

As described above, the alarm 215 may spur the patient to seek immediate medical attention to correct the problem, thereby limiting the potential complications of prolonged exposure to a malfunctioning IOP control system. In one embodiment, the accessory device 214 uploads the relevant data to a remote accessible data storage site (e.g., an internet server, email server, text message server) that activates an alarm at the remote site. The relevant data may be uploaded to a remote accessible data storage site and the remote alarm may be activated so that it can be observed in real time, for example, by healthcare professionals that are not present at the patient's side. Thus, both the patient and his or her healthcare professionals may be alerted to a problem with the IOP control system, such as a potential blockage or clog within the IOP control system or a ruptured drainage site, prior to the patient's next scheduled examination.

Embodiments in accordance with the present disclosure provide users with a convenient and continuous clog detection system for their IOP control device that enables users to seek immediate medical attention for problematic flow control implants. Prompt awareness on the part of the user and/or the health care provider to an ineffective and/or clogged flow control system may result in better treatment, less complications, a more consistent recovery, and ultimately a better patient outcome.

Embodiments in accordance with the present disclosure may be used in a variety of applications to regulate flow and/or pressure. For example, but not by way of limitation, embodiments of the present disclosure may be utilized to detect abnormalities such as clogs in a flow control system as part of a microanalytical system, a dialysis system, a process control system, a drug delivery system, a solar thermal system, a cooling system, and/or a heating system. Some embodiments of the present disclosure may be utilized to detect abnormalities such as clogs in a variety of fluidic systems such as, but not by way of limitation, the urinary tract, the brain (e.g., to regulate intracranial pressure), and the circulatory/renal system (e.g., as part of a dialysis system). Moreover, some embodiments are shaped and configured for implantation in a patient, while others are not.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure

1. An apparatus for treatment of a medical condition of a patient to monitor drainage from a first region of a patient to a second region, comprising:
a first pressure sensor configured to measure a first pressure of the first region;
a second pressure sensor configured to measure a second pressure of the second region;
a flow system to regulate drainage of fluid from the first region to the second region;
a memory having a stored pressure drop threshold across the flow system; and
a processor associated with the memory and configured to compare the first pressure, the second pressure, and the stored pressure drop threshold and selectively generate control signals based on the comparisons.

2. The apparatus of claim 1, wherein the processor is configured to calculate a pressure differential between the first pressure and the second pressure and compare the pressure differential with the stored pressure drop threshold to determine whether the pressure differential is within an acceptable range.

3. The apparatus of claim 2, further comprising an alarm, wherein the control signals trip the alarm, the processor being configured to generate the control signals when the processor determines that the pressure differential is not within an acceptable range.

4. The apparatus of claim 3, further comprising an accessory device apart from the flow system, wherein the alarm is carried on the accessory device.

5. The apparatus of claim 4, wherein the processor is disposed in the accessory device.

6. The apparatus of claim 2, wherein the stored pressure drop threshold comprises an upper pressure drop threshold.

7. The apparatus of claim 6, further comprising an alarm, wherein the control signals trip the alarm, the processor being configured to determine that the pressure differential is not within an acceptable range and generate the control signals when the pressure differential exceeds the upper pressure drop threshold when the flow control system is in an open condition.

8. The apparatus of claim 2, wherein the stored pressure drop threshold comprises a lower pressure drop threshold.

9. The apparatus of claim 8, further comprising an alarm, wherein the control signals trip the alarm, the processor being configured to determine that the pressure differential is not within an acceptable range and generate the control signals when a pressure drop is less than the lower pressure drop threshold when the flow control system is in an open condition.

10. The apparatus of claim 2, wherein the stored pressure drop threshold comprises an upper pressure drop threshold and a lower pressure drop threshold.

11. The apparatus of claim 10, further comprising an alarm, wherein control signals trip the alarm, the processor being configured to determine that the pressure differential is not within an acceptable range and generate the control signals if the pressure differential between the first and second pressures is not less than the upper pressure drop threshold and greater than the lower pressure drop threshold when the flow control system is in an open condition.

12. The apparatus of claim 12, wherein the memory stores a predetermined time interval, and the processor is configured to compare a first pressure differential between the first and second pressures measured at a first time with the stored pressure drop threshold and to compare a second pressure differential between the first and second pressures measured at a second time with the stored pressure drop threshold, wherein the first and second times are separated by the predetermined time interval.

13. The apparatus of claim 2, further comprising an alarm, wherein control signals trip the alarm, the memory stores a predetermined time interval, and the processor is configured to generate the control signals if the first pressure does not change while the flow system is in an open condition for a time equal to or greater than the predetermined time interval.

14. The apparatus of claim 2, further comprising an alarm, wherein control signals trip the alarm, the memory stores a predetermined time interval, and the processor is configured to generate the control signals if the first pressure does not change and the pressure differential does not change while the flow system is in an open condition for a time equal to or greater than the predetermined time interval.

15. The apparatus of claim 1, wherein the flow system comprises one of a valve and a pump.

16. The apparatus of claim 1, further comprising an implantable medical device for treating an ocular condition, wherein the processor is carried on the implantable medical device.

17. The apparatus of claim 16, wherein the processor is configured to calculate an IOP of an eye based on measurements from the first pressure sensor and the second pressure sensor.

18. The apparatus of claim 16, further comprising a drainage tube connected to the flow system and sized to extend from the anterior chamber of an eye to the second region.

19. The apparatus of claim 18, wherein the processor is configured to compare a pressure differential between the first and second pressures with the stored pressure drop threshold and generate the control signals if the pressure differential is not within an acceptable range defined by the stored pressure drop threshold.

20. A control system for treatment of an ocular condition of a patient to ensure drainage from an anterior chamber of the eye to a drainage location, comprising:
a first pressure sensor configured to detect a pressure representative of an anterior chamber of the eye;
a second pressure sensor configured to detect a pressure representative of the drainage location;
a flow system to regulate drainage of fluid from the anterior chamber to the drainage location;
a memory having a stored pressure drop threshold across the flow system, the stored pressure drop threshold comprising an upper pressure drop threshold and a lower pressure drop threshold; and
a processor associated with the memory and configured to compare the first pressure, the second pressure, and the stored pressure drop threshold and selectively generate control signals based on the comparisons.

21. The control system of claim 20, further comprising an alarm, wherein the control signals trip the alarm, the processor being configured to generate the control signals when a pressure drop across the flow system is outside an acceptable range defined by the upper pressure drop threshold and the lower pressure drop threshold.

22. The control system of claim 21, further comprising an accessory device apart from the flow system, wherein the alarm is carried on the accessory device.

23. The control system of claim 22, further comprising a data transmission module structurally configured to receive data from the first and second pressure sensors and transmit the data between the sensors, the processor, the memory, and the accessory device.

24. The control system of claim 20, wherein the processor is configured to generate control signals when a pressure differential between the first and second pressures exceeds the upper pressure drop threshold when the flow control system is in an open condition.

25. The control system of claim 20, wherein the processor is configured to generate control signals when a pressure differential between the first and second pressures is less than the lower pressure drop threshold when the flow control system is in an open condition.

26. The control system of claim 24, wherein the memory stores a predetermined time interval, and the processor is configured to compare a first pressure differential between the first and second pressures measured at a first time with the stored pressure drop threshold and to compare a second pressure differential between the first and second pressures measured at a second time with the stored pressure drop threshold, wherein the first and second times are separated by the predetermined time interval.

27. The control system of claim 20, wherein the flow system comprises one of a valve and a pump.

28. The control system of claim 20, further comprising an implantable medical device for treating an ocular condition, wherein the processor is carried on the implantable medical device.

29. The control system of claim 20, further comprising a drainage tube connected to the flow system and sized to extend from the anterior chamber to the drainage location.

30. The control system of claim 28, further comprising a third pressure sensor configured to detect a pressure representative of atmospheric pressure, wherein the memory stores an IOP setpoint and a predetermined time threshold, the processor being configured to compare the first pressure, the third pressure, and the IOP setpoint and selectively generate control signals based on the comparisons.

31. The control system of claim 30, wherein the processor is configured to calculate a difference between the IOP setpoint and an IOP of an eye and to generate control signals if the IOP of the eye is less than the IOP setpoint for a time greater than the predetermined time threshold.

32. A method comprising:
storing a pressure drop threshold in a memory;
measuring a first pressure representative of a first region of an eye;
measuring a second pressure representative of a second region of an eye;
communicating the first and second pressures to a processor;
calculating a pressure differential between the first and second pressures with the processor;
comparing the pressure differential and the stored pressure drop threshold; and
activating an alarm when the pressure differential is outside an acceptable range defined by the pressure drop threshold.

33. The method of claim 32, wherein the pressure drop threshold comprises an upper pressure drop threshold and a lower pressure drop threshold.

34. The method of claim 33, wherein the acceptable range defined by the pressure drop threshold is bounded by the lower pressure drop threshold and the upper pressure drop threshold.

35. The method of claim 33, wherein activating the alarm comprises activating the alarm when the pressure differential exceeds the upper pressure drop threshold or when the pressure differential is less than the lower pressure drop threshold.

36. The method of claim 33, further comprising:
storing a predetermined time interval in the memory, and
evaluating if the pressure differential exceeds the upper pressure drop threshold or is less than the lower pressure drop threshold over the predetermined time interval.

37. The method of claim 32, comprising activating a flow control system to regulate fluid to flow from the first region to the second region.

38. The method of claim 37, comprising adjusting the stored pressure drop threshold while activating the flow control system.

39. The method of claim 37, further comprising receiving an input from a health care provider to adjust the stored pressure drop threshold while activating the flow control system.

40. The method of claim 37, further comprising powering off the flow control system when the alarm is activated.
